(19) United States
(12) Patent Application Publication
(10) Pub. No.: US 2026/0256804 A1
(43) Pub. Date: Sep. 3, 2026

(54) NOVEL PHARMACEUTICAL COMPOSITIONS AND METHODS FOR TREATING ANXIETY, DEPRESSION AND/OR OTHER PSYCHIATRIC DISORDERS

(71) Applicant: LA PharmaTech Inc., Blacksburg, VA (US)

(72) Inventors: Jianmin Wang, Blacksburg, VA (US); Fengming Jia, Beijing (CN); Geping Cui, Beijing (CN)

(21) Appl. No.: 19/066,937

(22) Filed: Feb. 28, 2025

(51) Int. Cl. A61K 31/55; A61K 31/135; A61P 25/22; A61P 25/24

(52) U.S. Cl. CPC A61K 31/55; A61K 31/135; A61P 25/22; A61P 25/24

(57) ABSTRACT

Pharmaceutical compositions comprising azelastine and sertraline are disclosed. Methods of using the pharmaceutical compositions for treating patients suffering from anxiety or depression, one or more psychiatric disorders such as anxiety, depression, major depressive disorder, generalized anxiety disorder, mild chronic depression, premenstrual dysphoric disorder, seasonal affective disorder, sleep disorder related to anxiety, are also disclosed.

### FIELD OF THE INVENTION

The invention relates to the field of practical medicine, namely, to the use of pharmaceutical compositions for treating anxiety, depression and/or alleviating manifestations of one or more psychiatric disorders, such as major depressive disorder, generalized anxiety disorder, mild chronic depression, premenstrual dysphoric disorder, and sleep disorder related to anxiety.

### BACKGROUND OF THE INVENTION

Anxiety, depression, panic disorder, and agitation, affect more than one in ten people globally (10.7%) as reported by the Institute for Health Metrics and Evaluation in their flagship Global Burden of Disease study in 2017. Two examples of psychiatric disorders having such symptoms, major depressive disorders (MDDs) and generalized anxiety disorder (GAD), are common, severe, chronic and often life-threatening illnesses. More than 20% of the adult population suffers from these conditions at some time during their life. Suicide is estimated to be a cause of death in up to approximately 15% of individuals with MDDs. In addition, MDDs represent a major risk factor for the development of cardiovascular disease and death after myocardial infarction.

Treatments for anxiety, depression and the like include selective serotonin reuptake inhibitors (SSRIs), such as citalopram (Celexa), escitalopram oxalate (Lexapro), fluoxetine (Prozac), fluvoxamine (Luvox), paroxetine HRI (Paxil), and sertraline (Zoloft); selective serotonin & norepinephrine inhibitors (SNRIs), such as desvenlafaxine (Khedezla), desvenlafaxine succinate (Pristiq), duloxetine (Cymbalta), levomilnacipran (Fetzima), and venlafaxine (Effexor); tetracyclic antidepressants of noradrenergic and specific serotonergic antidepressants (NaSSAs), such as Remeron; tricyclic antidepressants, such as Elavil, imipramine (Tofranil), nortriptyline (Pamelor), and Sinequan; monoamine oxidase inhibitors (MAOIs), such as isocarboxazid (Marplan), phenelzine (Nardil), selegiline (EMSAM), and tranylcypromine (Parnate); and benzodiazepines, such as alprazolam (Xanax), diazepam (Valium), buspirone (Buspar), and lorazepam (Ativan). These drugs do carry a risk of addiction, tolerance, loss of effectiveness, inciting violent or self-destructive actions, fatigue, and drowsiness along with nausea, increased appetite and weight gain, loss of sexual desire and other sexual problems, insomnia, dry mouth, blurred vision, and so on.

Sertraline is an antidepressant medication within the selective serotonin reuptake inhibitors (SSRIs) class with primarily inhibitory effects on presynaptic serotonin reuptake. This inhibition of serotonin reuptake results in an accumulation of serotonin. Serotonin in the central nervous system plays a role in regulating mood, personality, and wakefulness, which is why blocking serotonin reuptake is beneficial in disorders such as major depression. While the primary mechanisms of action in SSRIs are similar, Sertraline has unique pharmacokinetics, pharmacodynamics, and side effect profile.

The therapeutic effects of Sertraline cannot be entirely summed up by simple inhibition of the serotonin transporter (SERT), and as such other mechanisms of action must be at work. A current theory posits that the neuronal stress caused by sertraline causes a shift in brain homeostasis that results in downregulation of SERTs in some areas of the brain and upregulation in others. The serotonin transporter is organized as a component of a molecular complex which includes enzyme and several binding sites and localized on presynaptic axon terminals of serotonin neurons as well as on the cell bodies of serotonin neurons. When sertraline binds to the transporter, serotonin accumulates in the synapse, since its release is no longer accompanied by presynaptic transport back into the neuron. Those molecular events occur immediately after administration of sertraline. When the serotonin transporter is blocked by sertraline, serotonin no longer is transported out of the synapse, so accumulates there to prolong and increase its interactions at all the various serotonin receptor subtypes in the synapse including 5HT1A and 5HT2A receptors. But immediate actions of sertraline are mostly side effects due to the initiating actions of sertraline, such as negative allosteric modulation of the serotonin transporter. It was generally believed that serotonin is increased at specific serotonin receptor subtypes in discrete regions of the body where the relevant physiologic processes are regulated which explains these immediate side effects caused by sertraline.

When prescribing sertraline to any patient, a risk-benefit analysis must be evaluated with the potential treatment effects, adverse effects, and dangers of the illness to be treated. Many adverse effects are shared among all SSRIs to varying degrees, including nausea, vomiting, insomnia, drowsiness, headache, decreased sex drive, and agitation, prolonged QT interval, xerostomia. extrapyramidal symptoms (EPS), serotonin syndrome, rash, birth defects, hyponatremia, and cataracts.

Clearly, new treatments are highly and urgently needed that have tremendous improvement and significant impact to the management of patients with anxiety, depression and/or other psychiatric disorders, such as treatments being more effective and with fewer adverse effects.

Increasing evidence indicates that inflammatory processes can cause and contribute to the development of anxiety, depression and/or other psychiatric disorders. Inflammation can be defined as one of the immune responses for protecting living organisms from damage. The immune system can be triggered by various factors such as pathogens, damage to cells and stress that may induce acute or chronic inflammatory responses in organs including the brain, potentially leading to tissue damage or disease. The latest advancements in neurobiological research provide increasing evidence that inflammatory and neurodegenerative pathways play a relevant role in depression and anxiety. Preclinical and clinical studies on depression and anxiety highlighted an increased production of inflammatory markers, such as interleukin (IL)-1, IL-6, tumor necrosis factor (TNF)-αand interferon (INF)-αand γ, and overactivated inflammatory signaling pathways including nuclear factor kappa B (NF-κB). Other studies show that acute and chronic administration of cytokines or cytokine inducers were found to trigger depressive and/or anxiety symptoms. According to the cytokine hypothesis, depression and anxiety would be due to a stress-related increased production of pro-inflammatory cytokines that, in turn, would lead to increased oxidative and nitrosative brain damage and consequent reduced availability of tryptophan and serotonin (5-HT). Cytokines would also play a role in the onset of the glucocorticoid resistance, underlying the overdrive of the hypothalamic-pituitary-adrenal axis. Therefore, activation of the inflammatory and neurodegenerative pathways would lead to the brain damage observed in depression and/or anxiety through both reduced neurogenesis and increased neurodegeneration.

Azelastine is classified pharmacologically as a second-generation antihistamine and is a relatively selective, non-sedative, competitive antagonist at HI receptors for treatment of allergic rhinitis and asthma. But, more uniquely, its inhibition of inflammatory mediators and its mast cell stabilizing effects, in addition to its antihistaminic activity, place it among the new generation of dual-acting anti-inflammatory drugs. Its ability to modify several other mediators of inflammation, such as IL-1, IL-6, TNF-α and INF-α, and to reduce overactivation of NF-κB inflammatory signaling pathway might contribute to its mechanism of action of potential treatment of psychiatric disorders, such as anxiety, depression, panic disorder, agitation, bipolar disorder (BD), premenstrual dysphoric disorder (PDD). In vitro and in vivo studies, as well as clinical trials support the dual effects of direct inhibition and stabilization of inflammatory cells. In vitro data indicate that azelastine's affinity for inhibition of mast cell degranulation may also decrease the release of other inflammatory mediators, including leukotrienes and interleukin-1β, among others. Preclinical studies show that azelastine also directly antagonizes other mediators of inflammation, such as tumor necrosis factor-a, leukotrienes, endothelin-1, and platelet-activating factor.

Therefore, combination treatments comprising azelastine (antihistamine agent with anti-inflammatory activities) with sertraline (with inhibitory effects on presynaptic serotonin reuptake) would potentially provide, in terms of working through multi-mechanisms of actions, more effective treatments for depression, anxiety and/or psychiatric disorders, such as but not limited to major depressive disorder, generalized anxiety disorder, mild chronic depression, premenstrual dysphoric disorder, and sleep disorder related to anxiety, with less side effects.

### SUMMARY OF THE INVENTION

The present invention includes a pharmaceutical composition that comprises two active pharmaceutical ingredients. This pharmaceutical composition comprises the first active ingredient that is azelastine and the second active ingredient that is sertraline.

In some embodiments of this invention, azelastine (azelastine hydrochloride salt and/or other salt thereof) in the pharmaceutical composition is provided in an amount of about 1 mg to about 8 mg and sertraline (sertraline hydrochloride salt and/or other salt thereof) in an amount of about 25 mg to about 200 mg.

The present invention also includes an oral pharmaceutical dosage form of the pharmaceutical composition that is a solid, liquid, gel, or solution form.

The present invention further includes use of the composition, such as by oral dosage, through administration to patients for treating one or more of anxiety, depression, major depressive disorder (MDD), generalized anxiety disorder (GAD), mild chronic depression (MCD), bipolar disorder, premenstrual dysphoric disorder (PDD), and sleep disorders related to anxiety (SDRA).

In some embodiments of this invention, an oral pharmaceutical dosage form of the pharmaceutical composition containing azelastine (azelastine hydrochloride and/or other salt thereof) in an amount of about 1 mg to about 8 mg and sertraline (sertraline hydrochloride and/or other salt thereof) in an amount of about 25 mg to about 200 mg is administered to patients with anxiety, depression, or any one or more psychiatric disorder, such as MDD, GAD, MCD, PDD, SDRA.
Embodiments include Aspect 1, which are pharmaceutical compositions comprising: azelastine and sertraline; and one or more pharmaceutically acceptable excipients.
Aspect 2 is the pharmaceutical composition of Aspect 1, wherein the azelastine is present in the pharmaceutical composition in an amount in the range of about 1 mg to about 8 mg.
Aspect 3 is the pharmaceutical composition of Aspect 1 or 2, wherein the sertraline is present in the pharmaceutical composition in an amount in the range of about 25 mg to about 200 mg.
Aspect 4 is the pharmaceutical composition of any of Aspects 1-3, wherein the sertraline is present in the pharmaceutical composition in an amount in the range of about 25 mg to about 150 mg.
Aspect 5 is the pharmaceutical composition of any of Aspects 1-4, wherein: the azelastine is present in the pharmaceutical composition in an amount in the range of about 1 mg to about 6 mg; and the sertraline is present in the pharmaceutical composition in an amount in the range of about 50 mg to about 150 mg.
Aspect 6 is the pharmaceutical composition of any of Aspects 1-5, wherein the pharmaceutical composition is formulated as an oral pharmaceutical dosage form.
Aspect 7 is the pharmaceutical composition of any of Aspects 1-6, wherein the oral pharmaceutical dosage form is a solid form or a liquid form.
Aspect 8 is a method comprising: administering a pharmaceutical composition to a patient; wherein the pharmaceutical composition comprises effective amounts of azelastine and sertraline; and wherein the effective amounts together are sufficient to treat anxiety and/or depression and/or one or more psychiatric disorder, one or more symptoms of one or more psychiatric disorder, one or more disease, one or more diseases state, or one or more symptom of a disease or condition of the patient, such as anxiety, depression and/or sleep disorders, i) optionally wherein the effective amounts include any of 1 mg, 2 mg, 3 mg, 4 mg, 5 mg, 6 mg, 7 mg, 8 mg, 9 mg, 10 mg, 11 mg, or 12 mg azelastine or any salt thereof (such as HCl) and up to and including any of 25 mg, 50 mg, 75 mg, 100 mg 125 mg, 150 mg, 200 mg sertraline, or any salt thereof (such as HCl), or any amount within any of these ranges, ii) such as azelastine (or a salt thereof, such as HCl) in an amount in the range of about 2 mg to 6 mg, or 4 mg to 8 mg, or 1 mg to 6 mg, or 2 mg to 8 mg, or 4 mg to 6 mg in combination with sertraline (or a salt thereof, such as HCl) in an amount in the range of about 25 mg to about 200 mg, such as 25 mg to 150 mg, or 25 mg to 100 mg, or 25 mg to 50 mg, or 50 mg to 100 mg, or 50 mg to 150 mg, or 50 mg to 200 mg, or 100 mg to 150 mg, or 150 mg to 200 mg, or iii) such as 1 mg or 2 mg or 4 mg or 6 mg or 8 mg azelastine (or salt thereof, such as HCl) in combination with 25 mg or 50 mg or 100 mg or 150 mg or 200 mg sertraline (or salt thereof, such as HCl), or iv) such as azelastine (or salt thereof, such as HCl) and sertraline (or salt thereof, such as HCl) in synergistic amounts.
Aspect 9 is the method of Aspect 8, wherein one or more of the psychiatric disorders are selected from MDD, GAD, MCD, PDD, SDRA, and optionally wherein the patient has one or more of anxiety, depression, one or more psychiatric disorder (e.g., MDD, GAD, MCD, PDD, and/or SDRA), one or more symptoms of one or more psychiatric disorder, one or more disease, one or more diseases state, or one or more symptom of a disease or condition of the patient, such as anxiety, depression and/or sleep disorders.
Aspect 10 is the method of Aspect 8 or 9, wherein the pharmaceutical composition is administered once or twice a day, or once every 2 or 3 or 4 days to the patient in an oral solid or liquid form.
Aspect 11 is the method of any of Aspects 8-10, wherein the pharmaceutical composition is administered for a period of at least 2 weeks.
Aspect 12 is the method of any of Aspects 8-11, wherein the azelastine is present in the pharmaceutical composition in an amount in the range of about 1 mg to about 8 mg.
Aspect 13 is the method of any of Aspects 8-12, wherein the sertraline is present in the pharmaceutical composition in an amount in the range of about 25 mg to about 200 mg.
Aspect 14 is the method of any of Aspects 8-13, wherein the sertraline is present in the pharmaceutical composition in an amount in the range of about 50 mg to about 150 mg.
Aspect 15 is the method of any of Aspects 8-14, wherein the azelastine is present in the pharmaceutical composition in an amount in the range of about 2 mg to about 6 mg.
Aspect 16 is the method of any of Aspects 8-15, wherein: the azelastine is present in the pharmaceutical composition in an amount in the range of about 2 mg to about 8 mg; and the sertraline is present in the pharmaceutical composition in an amount in the range of about 50 mg to about 150 mg.

### DETAILED DESCRIPTION OF THE INVENTION

Through clinical practice, the inventors of the present invention found that a pharmaceutical composition with oral dosage forms comprising the active agents, a salt form of azelastine and sertraline, is suitable for treating patients suffering from psychiatric disorders such as one or more of major depressive disorder (MDD), generalized anxiety disorder (GAD), mild chronic depression (MCD), bipolar disorder, premenstrual dysphoric disorder (PDD), and sleep disorders related to anxiety (SDRA).

The detailed description provided below is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

Definitions As used in the present specification, the following words and phrases are generally intended to have the meanings as set forth below, except to the extent that the context in which they are used indicates otherwise.

Psychiatric disorders can include but are not limited to anxiety, depression, MDD, GAD, MCD, PDD, SDRA.

As used herein, the term “sertraline” refers to sertraline free base, (1S-cis)-4-(3,4-dichlorophenyl)-1,2,3,4-tetrahydro-N-methyl-1-naphthalenamine. In certain embodiments, sertraline also includes any pharmaceutically acceptable salt, such as the hydrochloride or HCl salt. Preferably, in any embodiment of the invention as described herein, sertraline is in the form of its hydrochloride salt, as sertraline hydrochloride or sertraline HCl. More preferably, in any embodiment of the invention as described herein, reference to the amounts and dosage ranges of sertraline in the solid oral dosage forms are to the amounts and dosage ranges of sertraline hydrochloride.

As used herein, the term “azelastine” refers to azelastine free base, or 4-(p-Chlorobenzyl)-2-(hexahydro-1-methyl-1H-azepin-4-yl)-1-(2H)-phthalazinone. In certain embodiments, azelastine also includes any pharmaceutically acceptable salt, such as the hydrochloride or HCl salt. Preferably, in any embodiments of the invention as described herein, azelastine is in the form of its hydrochloride salt, as azelastine hydrochloride or azelastine HCl. More preferably, in any embodiment of the invention as described herein, reference to the amounts and dosage ranges of azelastine in the solid oral dosage forms are to the amounts and dosage ranges of azelastine hydrochloride.

As used herein, “treating” or “treatment” means complete cure or incomplete cure, or it means that the symptoms of the underlying disease or associated conditions are at least reduced and/or delayed, and/or that one or more of the underlying cellular, physiological, or biochemical causes or mechanisms causing the symptoms are reduced, delayed and/or eliminated. It is understood that reduced or delayed, as used in this context, means relative to the state of the untreated disease, including the molecular state of the untreated disease, not just the physiological state of the untreated disease.

The term “effective amount” refers to an amount that is sufficient to affect treatment, as defined below, when administered to a mammal in need of such treatment. The therapeutically effective amount will vary depending upon the patient being treated, the weight and age of the patient, the severity of the disease condition, the manner of administration and the like, which can readily be determined by one of ordinary skill in the art. The pharmaceutical compositions may be administered in either single or multiple doses by oral administration. Administration may be by way of any one or more of capsule, tablet, gel, spray, drops, solution, suspensions, syrups, or the like.

The term “about” used herein in the context of quantitative measurements means the indicated amount ±10%. For example, with a ±10% range, “about 2 mg” can mean 1.8-2.2 mg.

The pharmaceutical compositions may be formulated for pharmaceutical use using methods known in the art, for example, Ansel's Pharmaceutical Dosage Forms and Drug Delivery Systems Tenth (by Loyd Allen, 2013) and Handbook of Pharmaceutical Manufacturing Formulations (Volumes 1-6 by Sarfaraz K. Niazi). Accordingly, incorporation of the active compounds and a controlled, or slow-release matrix may be implemented.

Either fluid or solid unit dosage forms can be readily prepared for oral administration, for example, admixed with any one or more of conventional ingredients such as dicalcium phosphate, magnesium aluminum silicate, magnesium stearate, calcium sulfate, starch, talc, lactose, acacia, methyl cellulose and functionally similar materials as pharmaceutical excipients or carriers. A sustained release formulation may optionally be used. In older or incoherent subjects sustained release formulations may even be preferred. Capsules may be formulated by mixing the pharmaceutical composition with a pharmaceutical diluent which is inert and inserting this mixture into a hard gelatin capsule having the appropriate size. If soft capsules are desired, a slurry of the pharmaceutical composition with an acceptable vegetable, light petroleum or other inert oil can be encapsulated by forming into a gelatin capsule.

Suspensions, syrups and elixirs may be used for oral administration or fluid unit dosage forms. A fluid preparation including oil may be used for oil soluble forms. A vegetable oil such as corn oil, peanut oil or a flower oil, for example, together with flavoring agents, sweeteners and any preservatives produces an acceptable fluid preparation. A surfactant may be added to water to form a syrup for fluid unit dosages. Hydro-alcoholic pharmaceutical preparations may be used having an acceptable sweetener, such as sugar, saccharin or other non-nutritive sweetener, and/or a biological sweetener and/or a flavoring agent, such as in the form of an elixir.

The solid oral dosage formulation of this disclosure means a form of tablets, caplets, bi-layer tablets, film-coated tablets, pills, capsules, or the like. Tablets in accordance with this disclosure can be prepared by any mixing and tableting techniques that are well known in the pharmaceutical formulation industry. In some examples, the dosage formulation is fabricated by direct compressing the respectively prepared sustained-release portion and the immediate-release portion by punches and dies fitted to a rotary tableting press, ejection or compression molding or granulation followed by compression.

The pharmaceutical compositions provided in accordance with the present disclosure can be typically administered orally. This disclosure therefore provides pharmaceutical compositions that comprise a solid dispersion comprising azelastine and sertraline as described herein and one or more pharmaceutically acceptable excipients or carriers including but not limited to, inert solid diluents and fillers, diluents, including sterile aqueous solution and various organic solvents, permeation enhancers, solubilizers, disintegrants, lubricants, binders, glidants, adjuvants, and combinations thereof. Such compositions are prepared in a manner well known in the pharmaceutical arts (see, e.g., Ansel's Pharmaceutical Dosage Forms and Drug Delivery Systems, Tenth (by Loyd Allen, 2013) and Handbook of Pharmaceutical Manufacturing Formulations (Volumes 1-6 by Sarfaraz K. Niazi)).

The pharmaceutical compositions may further comprise pharmaceutical excipients such as diluents, binders, fillers, glidants, disintegrants, lubricants, solubilizers, and combinations thereof. Some examples of suitable excipients are described herein. When the pharmaceutical compositions are formulated into tablets, tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In embodiments, the pharmaceutical compositions can comprise a) about 1 mg 8 mg of azelastine (such as HCl (or other salt thereof)) and b) about 25 mg to 200 mg of sertraline (such as HCl or other salt thereof), or any amount of azelastine or sertraline (or salt thereof, such as HCl) within these ranges. Additional embodiments include pharmaceutical compositions comprising a) about up to and including any of 1 mg, 2 mg, 3 mg, 4 mg, 5 mg, 6 mg, 7 mg, 8 mg, 9 mg, 10 mg, 11 mg, or 12 mg azelastine or any salt thereof (such as HCl), and b) about up to and including any of 25 mg, 50 mg, 75 mg, 100 mg 125 mg, 150 mg, 200 mg sertraline, or any salt thereof (such as HCl), or any amount within any of these ranges. For example, the compositions can comprise a) about 2 mg of azelastine HCl and b) about 25 mg of sertraline HCl. Further, for example, compositions of the invention can comprise a) azelastine or any salt thereof (such as HCl) present in an amount in the range of about 1 mg to about 12 mg, such as 2 mg to 6 mg, or 4 mg to 8 mg, or 1 mg to 6 mg, or 2 mg to 8 mg, or 4 mg to 6 mg, and sertraline or any salt thereof (such as HCl) in an amount in the range of about 25 mg to about 200 mg, such as 25 mg to 150 mg, or 25 mg to 100 mg, or 25 mg to 50 mg, or 50 mg to 100 mg, or 50 mg to 150 mg, or 50 mg to 200 mg, or 100 mg to 150 mg, or 150 mg to 200 mg. Even further, compositions of embodiments of the invention include compositions comprising 1 mg or 2 mg or 4 mg or 6 mg or 8 mg azelastine or any salt thereof (such as HCl) in combination with 25 mg or 50 mg or 100 mg or 150 mg or 200 mg sertraline or any salt thereof (such as HCl). In embodiments, azelastine and sertraline (or any salt thereof, such as HCl), are present in the compositions in synergistic amounts. In embodiments, the amount of azelastine (or any salt thereof, such as HCl) present in the composition can be equal to, more than, or less than the amount of sertraline (or any salt thereof, such as HCl) present in the composition. In embodiments, the amount of sertraline (or any salt thereof, such as HCl) present in the composition can be 2 or 3 times as much, or 4 times as much, or 5, 6, 7, 8, 9, 10, 15, 20, 25, 30, 35, 40, 45, or 50 times as much as the amount of azelastine (or any salt thereof, such as HCl) present in the composition. Any one or more of the compositions of the invention can be used with any one or more the methods of the invention disclosed herein, or other methods of using the compositions.

It will be understood, that the amount of the pharmaceutical composition containing azelastine and sertraline actually administered usually will be determined by a physician, in the light of the relevant circumstances, including the condition to be treated, the chosen route of administration, the actual compound administered and its relative activity, the age, weight and response of the individual patient, the severity of the patient's symptoms, and the like.

The pharmaceutical compositions, pharmaceutical dosage forms, and tablets containing azelastine, such as azelastine, and sertraline as described herein are administered to a patient suffering from one or more psychiatric disorders such as anxiety, depression, MDD, GAD, MCD, PDD, SDRA, by administration (such as oral administration) once daily, twice daily, up to four times a day, once every other day, once a week, two times a week, three times a week, four times a week, or five times a week, or combinations thereof, for a period of about 1 week, or a period of about 1-2 weeks, 1-3 weeks, 2-4 weeks, 2-6 weeks, 1-8 weeks, 2-8 weeks, 4 weeks, 5 weeks, 3-6 weeks, 6 weeks, 7 weeks, 6-8 weeks, 8-12 weeks, or any period in between.

In embodiments, patients are administered the pharmaceutical composition(s) with a therapeutic effective daily dosage of azelastine (or any salt thereof, such as HCl) in the range of about 1 mg to about 8 mg and sertraline (or any salt thereof, such as HCl) in an amount in the range of about 25 mg to about 200 mg.

In embodiments, the pharmaceutical dosage forms and tablets of pharmaceutical compositions containing azelastine and sertraline (or their salts thereof, such as HCl) as described herein are effective in reversing, reducing, alleviating, and/or treating anxiety or depression and/or one or more symptoms in patients with one or more psychiatric disorders such as anxiety, depression, MDD, GAD, MCD, PDD, SDRA in about 1-8 weeks, such as within 1, 2, 3, 4, 5, 6, 7, or 8 weeks, or any range in between.

The following Examples are illustrative and should not be interpreted to limit the scope of the claimed subject matter.

### EXAMPLE 1

A 54-year-old female patient was diagnosed with mild chronic depression (MCD) with constant sadness and lack of interest and feeling lonely and helpless, which are possible symptoms of MCD. She was prescribed daily doses of trazodone, bupropion, and sertraline (50 mg for 2 weeks, 100 mg for 3 weeks), respectively and with escalations of various doses (e.g., 150 mg or 200 mg sertraline) for 3 months from her 5 clinic visits. But the patient's symptoms were only improved by less than 20%. Then she was prescribed a daily dose with a combination of azelastine (2 mg) and sertraline (100 mg) for a treatment of 2 weeks. By the end of the 2nd week, she reported the improvement of her MCD symptoms by 50% with no side effects. Then she was prescribed with a combination of azelastine (4 mg) and sertraline (150 mg) for 3 weeks during her 7th clinic visit. By the end of the 5^{th} week of her treatment, she reported 90% improvement with no intolerable side effects. With 2 more weeks of a continued treatment with the combination of azelastine (4 mg) and sertraline (150 mg), her sadness, lack of interest, feeling lonely and helpless were in remission and she showed no MCD symptoms. Because azelastine is approved for the treatment of allergic rhinitis and asthma and has not been approved to treat patients with MCD, this dramatic clinical outcome from the composition was unexpected. Although the inventors do not intend to be bound by this theory, it is believed that azelastine's anti-inflammatory action by suppressing release of cytokines, such as IL-1, IL-6, TNF-α and INF-α, in the CNS system may help sertraline more effectively mediate stimulation of 5HT1A somatodendritic autoreceptors located in the midbrain raphe and facilitate desensitization and adaptations of 5HT1A, 5HT2A and 5HT1D receptors, especially in certain projection pathways in the brain, because adaptations in serotonergic functioning are linked to untreated depression which demonstrates nonresponse to sertraline treatment. Further, azelastine's anti-inflammatory action may help sertraline's actions in the areas of presynaptic axonal terminal and dendrites in more effective ways with the serotonergic pathways. This composition of two mechanisms of actions provides a new solution for treating MCD, MCD symptoms, and/or sadness, lack of interest, feeling lonely, or feeling helpless.

### EXAMPLE 2

A 47-year-old male patient was diagnosed with major depressive disorder (MDD) with constant sadness, lack of interest, and feeling lonely, helpless, and hopeless. Initially, he was prescribed sertraline (25 mg) daily but showed no improvement after a 2-week treatment. Then he was treated with 50 mg for a week and then 100 mg for 2 weeks. But the patient still had no improvement. Then he was prescribed with a daily dose comprising a combination of azelastine (2 mg) and sertraline (50 mg) starting in the 5^{th} week of his clinic visits. By the end of the 6^{th} week of his treatments, he reported improvement by 50% with no side effects. Then he was prescribed with a combination of azelastine (4 mg) and sertraline (100 mg) starting in the 7^{th} week of his clinic visits. By the end of the 9^{th} week of his treatments, his sadness, lack of interest, and feeling lonely, helpless, and hopeless were in remission and he had no symptoms of MDD. The inventors believe the hypothesis as explained in example 1 above is applicable here as well. In this case, the composition of two mechanisms of actions provided a new solution for treating his MDD, MDD symptoms, and/or sadness, lack of interest, feeling lonely, feeling helpless, or feeling hopeless, which could not be treated with other options, such traditional SSRIs and the like alone.

### EXAMPLE 3

A 29-year-old female patient was diagnosed with generalized anxiety disorder (GAD) but could not tolerate benzodiazepines, such as alprazolam, buspirone and MAOIs with initial 5 weeks of treatments from her clinic visits. Then she was treated with sertraline (50 mg) daily for 2 weeks and then 100 mg for 2 weeks, and lastly 200 mg for 2 weeks. But her GAD, and GAD symptoms, persisted while she complained about having nausea and diarrhea along with increased level of anxiety during the 5^{th} and 6^{th} weeks of her treatments with sertraline. Starting the 7^{th} week of her clinic visit, she was treated with the daily combination of azelastine (4 mg) and sertraline (50 mg). By the end of the first week of the treatment with the combination, the patient reported improvement in her anxiety without any complaint of side effects. With a continued treatment of the same combination for 3 more weeks, she reported 90% of reduction of her uncontrollable and persistent worry, fatigue, headaches, irritability, and nervousness (possible symptoms of GAD). The inventors believe the hypothesis as explained in example 1 above is applicable here as well. In this case, the composition of two mechanisms of actions provided a new solution for treating her GAD, her GAD symptoms, and/or her anxiety, uncontrollable and persistent worry, fatigue, headaches, irritability, and nervousness, which could not be treated with other options, such traditional SSRIs and MAOIs and the like alone.

### EXAMPLE 4

A 59-year-old female patient was diagnosed with MDD. The patient was treated with imipramine, bupropion, fluoxetine and sertraline respectively over 6 months of her clinic visits. But the patient's MDD symptoms were persistent, and she could not tolerate sertraline 200 mg daily treatment because of her increased level of anxiety, agitation and sweating. Then the patient was prescribed with the daily combination of azelastine (6 mg) and sertraline (50 mg) for a treatment for 2 weeks. By the end of the first treatment, the patient reported about 40% improvement. For the second treatment for 2 weeks, the patient was prescribed with the combination of azelastine (8 mg) and sertraline (100 mg). By the end of the second treatment, the patient reported about 90% improvement with no complaint of intolerable side effects. The inventors believe the hypothesis as explained in example 1 above is applicable here as well. In this case, the two compositions of two mechanisms of actions provided a new solution for treating her anxiety and agitation, her MDD, and MDD symptoms (such as anxiety, agitation and sweating), which could not be treated with other options.

### EXAMPLE 5

A 62-year-old male patient was diagnosed with MDD. Initial treatments with trazodone, bupropion, and sertraline from daily doses of 25 mg to 200 mg over 3 months of clinic visits failed because the patient's symptoms were persistent and the side effects of nausea, diarrhea, and anxiety from those medications were intolerable to the patient. He was prescribed azelastine (8 mg) daily for 2 weeks but there was no response although the dose was well tolerated. Then the patient was prescribed the daily combination of azelastine (8 mg) and sertraline (25 mg) for a treatment for 3 weeks. At the end of the first treatment with the combination, the patient reported about 50% improvement. For the second treatment, for 4 weeks the patient was prescribed the combination of azelastine (8 mg) and sertraline (50 mg). By the end of the second treatment with the new combination, the patient reported about 90% improvement with no complaint of intolerable side effects. The inventors believe the hypothesis in example 1 above is applicable here as well. In this case, the composition of two mechanisms of actions provided a new solution for treating anxiety, MDD and MDD symptoms which could not be treated with other options.

### EXAMPLE 6

A 45-year-old female patient was having chronic rhinitis when she was diagnosed with MDD. The patient was provided with daily azelastine (2 mg) for 2 weeks from her first clinic visit and azelastine (4 mg) for 3 weeks from her second clinic visit. By the second week of azelastine (4 mg) treatment, she reported no more symptoms of runny nose, itching, and sneezing caused by her chronic rhinitis but her persistent sadness and anxiety, feeling fatigued and lacking energy along with difficulty of sleeping, possible symptoms from MDD, were persistent. On the 8^{th} day after ending her azelastine (4 mg) treatment she was prescribed sertraline (25 mg) daily for 2 weeks during her 3^{rd} clinic visit and then sertraline (100 mg) for 2 weeks during her 4^{th} clinic visit. But the sadness and anxiety and feeling fatigued and lacking energy along with difficulty of sleeping were improved only by less than 20%, and she started complaining about side effects of nausea and diarrhea. Then the patient was prescribed with the daily combination of azelastine (4 mg) and sertraline (50 mg) for a treatment for 2 weeks during her 5^{th} clinic visit. By the end of the first treatment with the combination, she reported about 80% improvement in her sadness, anxiety, and fatigue and no difficulty of sleeping. After a continued treatment with the same composition for 3 more weeks, the patient reported no major symptoms of MDD. The inventors believe the hypothesis as explained in example 1 above is applicable here as well. In this case, the composition of two mechanisms of actions provided a new solution for treating sadness, anxiety, fatigue, lack of energy, and difficulty sleeping, and/or her MDD or MDD symptoms, which could not be treated with either azelastine alone or sertraline alone.

### EXAMPLE 7

A 37-year-old female patient was diagnosed with premenstrual dysphoric disorder (PMDD) complaining or the feelings of sadness, hopelessness, and worthlessness with increased level of anxiety and frequent tearfulness. The patient was provided with fluoxetine 40 mg daily for 2 weeks but there was no improvement. Then a prescription of sertraline 75 mg daily for 2 weeks not only failed to produce any improvement but also showed side effects of nausea and headache from the prescription. With her 3rd clinic visit, the patient was prescribed with the daily combination of azelastine (2 mg) and sertraline (50 mg) for 2 weeks. At the end of the first treatment with the combination, she reported about 50% improvement. After continued treatment with the same composition for 4 more weeks, the patient reported remission of her feelings of sadness, hopelessness, and worthlessness, anxiety and frequent tearfulness with no major symptoms of PMDD. The inventors believe the hypothesis as explained in example 1 above is applicable here as well. In this case, the composition of two mechanisms of actions provided a new solution for treating feelings of sadness, hopelessness, and worthlessness, anxiety, frequent tearfulness, her PMDD and symptoms thereof, which could not be treated with other options.

### EXAMPLE 8

A 61-year-old male patient had been having rhinitis with stuffy, runny and itch nose, endless sneezing, coughing, and itchy ears with repeated infections for more than 2 months when he was diagnosed with depression. The patient was prescribed azelastine (2 mg) daily for 2 weeks from his first clinic visit and azelastine (4 mg) daily for 2 weeks from his second clinic visit. By the end of the second week of azelastine (4 mg) treatment, the patient reported no symptoms of his previous rhinitis such as stuffy, runny and itch nose, excessive sneezing and coughing, and ear infections. But during his 3^{rd} clinic visit in the 2^{nd} week after the end of his azelastine treatment, he still complained about his depression, more worries, persistent sadness and anxiety, feeling exhausted and having no sex drive. He was prescribed with sertraline (50 mg) daily for 2 weeks but had no improvement. Then he was prescribed with sertraline (100 mg) daily for 2 weeks during her 4^{th} clinic visit. He improved by only less than 20%. Then the patient was prescribed with the daily combination of azelastine (2 mg) and sertraline (50 mg) for a treatment for 3 weeks during his 5^{th} clinic visit. By the end of the treatment with the combination, he reported about 80% improvement. After continued treatment with the same combination for 3 more weeks, the patient reported remission of his depression, feelings of worry, sadness and anxiety, feeling exhausted and having no sex drive, and reported no major symptoms of depression. The inventors believe the hypothesis as explained in example 1 above is applicable here as well. In this case, the composition of two mechanisms of actions provided a new solution for treating his depression, feelings of worry, sadness and anxiety, feeling exhausted and having no sex drive (possible symptoms of depression), which could not be treated with either azelastine alone or sertraline alone.

### EXAMPLE 9

A 70-year-old male patient was diagnosed with GAD as his relatives notified about his persistent inappropriate worries and he complained about nausea, headaches, sweating, muscle aches, and difficulty of falling asleep for 2 months. The patient was prescribed sertraline (25 mg) daily for 4 weeks but had no improvement. Then the patient was prescribed with the daily combination of azelastine (1 mg) and sertraline (25 mg) for a treatment for 4 weeks during his 2^{nd} clinic visit. By the end of the treatment with the combination, he reported about 80% improvement in persistent inappropriate worries, nausea, and difficulty of falling asleep. After a continued treatment with the same combination for 4 more weeks, the patient and his relatives reported remission of his feelings of worry, nausea, headaches, sweating, muscle aches, difficulty of falling asleep, and no major symptoms of GAD from the patient. The inventors believe the hypothesis as explained in example 1 above is applicable here as well. In this case, the composition of two mechanisms of actions provided a new solution for treating his GAD, feelings of worry, nausea, headaches, sweating, muscle aches, difficulty of falling asleep (possible symptoms of GAD), which could not be treated with sertraline alone.

### EXAMPLE 10

A 41-year-old female patient was diagnosed with premenstrual dysphoric disorder (PMDD) showing severe depression and extreme anxiety with complaining of extreme sad mood, frequent tearfulness, fatigue, anger, frequent headache and insomnia, and lack of energy before and during her regular periods. Both prescriptions of fluoxetine and sertraline (e.g., up to 200 mg/day) provided no relief, although no side effects were reported by the patient. Then the patient was prescribed with the daily combination of azelastine (8 mg) and sertraline (200 mg) for 3 weeks. At the end of the treatment with the composition, she reported about 60% improvement. After continued treatment with the same combination for 4 more weeks, the patient reported no depression, anxiety, extreme sad mood, frequent tearfulness, fatigue, anger, frequent headache and insomnia, such as anxiety-related insomnia. The inventors believe the hypothesis in example 1 above is applicable here as well. In this case, the composition of two mechanisms of actions provided a new solution for treating her PMDD, depression, anxiety, insomnia and anxiety-related sleep disorder (possible symptoms of PMDD), which could not be treated with other options.

### EXAMPLE 11

A male patient diagnosed with mild chronic depression (MCD) with constant sadness and lack of interest and feeling lonely and helpless, for example, would be prescribed daily amounts of azelastine (or salt thereof, such as HCl) in an amount in the range of 1-8 mg and sertraline (or salt thereof, such as HCl) in an amount in the range of 25-200 mg for a period of time in the range of about 1-12 weeks or longer, such as 1 mg azelastine and 25 mg sertraline for about 2-4 weeks. If the patient shows improvement, he can remain on this dosing indefinitely. If any of the sadness and lack of interest and feeling lonely and helpless, or his MCD symptoms persist, the dose (of each azelastine and sertraline) could be increased to 2 mg azelastine and 50 mg sertraline for 2-4 weeks. If the patient shows improvement, he can remain on this dosing indefinitely. If any of the sadness and lack of interest and feeling lonely and helpless, or his MCD symptoms persist, the dose could be increased to 4 mg azelastine and 100 mg sertraline for a period of 2-4 weeks. If the patient shows improvement, he can remain on this dosing indefinitely. If any of the sadness and lack of interest and feeling lonely and helpless, or his MCD symptoms persist, the dose could be increased to 8 mg azelastine and 200 mg sertraline for 2-4 weeks. If the patient shows improvement, he can remain on this dosing indefinitely. Or a practitioner can start at any combination dosage of azelastine and sertraline, increase, decrease or change the dosage as needed, and when an effective dose is established, the patient can continue treatment at the effective dose.

### EXAMPLE 12

A male patient diagnosed with mild chronic depression (MCD) with constant sadness and lack of interest and feeling lonely and helpless, for example, would be prescribed daily amounts of azelastine (or salt thereof, such as HCl) in an amount in the range of 2-6 mg and sertraline (or salt thereof, such as HCl) in an amount in the range of 25-200 mg for a period of time in the range of about 1-12 weeks or longer, such as 2 mg azelastine and 25 mg sertraline for 4 weeks. If the patient shows improvement, he can remain on this dosing indefinitely. If any of the sadness and lack of interest and feeling lonely and helpless, or his MCD symptoms persist, the dose (of azelastine and sertraline) could be increased to 4 mg azelastine and 50 mg sertraline for a period of 2-4 weeks. If the patient shows improvement, he can remain on this dosing indefinitely. If any of the sadness and lack of interest and feeling lonely and helpless his MCD symptoms persist, the amount of the sertraline dose could be increased (e.g., 4 mg azelastine and 200 mg sertraline) for a period of 2-4 weeks. If the patient shows improvement, he can remain on this dosing indefinitely. If any of the sadness and lack of interest and feeling lonely and helpless, or his MCD symptoms persist, the amount of the azelastine dose could be increased (e.g., 6 mg azelastine and 200 mg sertraline) for 2-4 weeks. If the patient shows improvement, he can remain on this dosing indefinitely. Or a practitioner can start at any combination dosage of azelastine and sertraline, increase, decrease or change the dosage as needed, and when an effective dose is established, the patient can continue treatment at the effective dose.

### EXAMPLE 13

A male patient is diagnosed with generalized anxiety disorder (GAD). The patient's complaints may include one or more of: anxiety, trouble concentrating, trouble falling asleep, trembling, twitching, tense muscles, headaches, grouchiness, sweating, hot flashes, lightheadedness, trouble breathing, nausea, fatigue, and/or being unable to relax. He, for example, would be prescribed once or twice daily the combination of azelastine (or salt thereof, such as HC1) in an amount in the range of 2-8 mg and sertraline (or salt thereof, such as HCl) in an amount in the range of 25-200 mg for a period of time in the range of about 1-12 weeks or longer, such as 2 mg azelastine and 25 mg sertraline for a period of 1-4 weeks. If the patient shows improvement, he can remain on this dosing indefinitely. If any of his anxiety, trouble concentrating, trouble falling asleep, trembling, twitching, tense muscles, headaches, grouchiness, sweating, hot flashes, lightheadedness, trouble breathing, nausea, fatigue, and/or being unable to relax, or his GAD symptoms persist, the amount of the sertraline dose could be increased (e.g., 2 mg azelastine and 50 mg sertraline) for 1-4 weeks. If the patient shows improvement, he can remain on this dosing indefinitely. If any of his anxiety, trouble concentrating, trouble falling asleep, trembling, twitching, tense muscles, headaches, grouchiness, sweating, hot flashes, lightheadedness, trouble breathing, nausea, fatigue, and/or being unable to relax, or his GAD symptoms persist, the dose (azelastine and sertraline) could be increased to 4 mg azelastine and 100 mg sertraline for a period of from 1-4 weeks. If the patient shows improvement, he can remain on this dosing indefinitely. If any of his anxiety, trouble concentrating, trouble falling asleep, trembling, twitching, tense muscles, headaches, grouchiness, sweating, hot flashes, lightheadedness, trouble breathing, nausea, fatigue, and/or being unable to relax, or his GAD symptoms persist, the dose (azelastine and sertraline) could be increased to 6 mg azelastine and 150 mg sertraline for 1-4 weeks. If the patient shows improvement, he can remain on this dosing indefinitely. If any of his anxiety, trouble concentrating, trouble falling asleep, trembling, twitching, tense muscles, headaches, grouchiness, sweating, hot flashes, lightheadedness, trouble breathing, nausea, fatigue, and/or being unable to relax, or his GAD symptoms persist, the dose (azelastine and sertraline) could be increased to 8 mg azelastine and 200 mg sertraline for 1-4 weeks. If the patient shows improvement, he can remain on this dosing indefinitely. Or a practitioner can start at any combination dosage of azelastine and sertraline, increase, decrease or change the dosage as needed, and when an effective dose is established, the patient can continue treatment at the effective dose.

### EXAMPLE 14

A female patient is diagnosed with generalized anxiety disorder (GAD). The patient's complaints may include one or more of: anxiety, trouble concentrating, trouble falling asleep, trembling, twitching, tense muscles, headaches, grouchiness, sweating, hot flashes, lightheadedness, trouble breathing, nausea, fatigue, and/or being unable to relax. She, for example, would be prescribed a daily amount of azelastine (or salt thereof, such as HCl) in an amount in the range of 1-8 mg and sertraline (or salt thereof, such as HCl) in an amount in the range of 25-50 mg for a period of time in the range of about 1-12 weeks or longer, such as 1 mg azelastine and 25 mg sertraline for a period of 1-4 weeks. If the patient shows improvement, she can remain on this dosing indefinitely. If any of the anxiety, trouble concentrating, trouble falling asleep, trembling, twitching, tense muscles, headaches, grouchiness, sweating, hot flashes, lightheadedness, trouble breathing, nausea, fatigue, and/or being unable to relax, or her GAD symptoms persist, the amount of the azelastine dose could be increased (e.g., 2 mg azelastine and 25 mg sertraline) for a period of 2-4 weeks. If the patient shows improvement, she can remain on this dosing indefinitely. If any of the anxiety, trouble concentrating, trouble falling asleep, trembling, twitching, tense muscles, headaches, grouchiness, sweating, hot flashes, lightheadedness, trouble breathing, nausea, fatigue, and/or being unable to relax, or her GAD symptoms persist, the dose (azelastine and sertraline) could be increased to 4 mg azelastine and 50 mg sertraline for a period of 2-4 weeks. If the patient shows improvement, she can remain on this dosing indefinitely. If any of the anxiety, trouble concentrating, trouble falling asleep, trembling, twitching, tense muscles, headaches, grouchiness, sweating, hot flashes, lightheadedness, trouble breathing, nausea, fatigue, and/or being unable to relax, or her GAD symptoms persist, the amount of the azelastine dose could be increased (e.g., 6 mg azelastine and 50 mg sertraline) for a period of 2-4 weeks. If the patient shows improvement, she can remain on this dosing indefinitely. If any of the anxiety, trouble concentrating, trouble falling asleep, trembling, twitching, tense muscles, headaches, grouchiness, sweating, hot flashes, lightheadedness, trouble breathing, nausea, fatigue, and/or being unable to relax, or her GAD symptoms persist, the azelastine dose could be increased again (e.g., 8 mg azelastine and 50 mg sertraline) for a period of 2-4 weeks. If the patient shows improvement, she can remain on this dosing indefinitely. Or a practitioner can start at any combination dosage of azelastine and sertraline, increase, decrease or change the dosage as needed, and when an effective dose is established, the patient can continue treatment at the effective dose.

### EXAMPLE 15

A female patient is diagnosed with generalized anxiety disorder (GAD). The patient's complaints may include one or more of: anxiety, trouble concentrating, trouble falling asleep, trembling, twitching, tense muscles, headaches, grouchiness, sweating, hot flashes, lightheadedness, trouble breathing, nausea, fatigue, and/or being unable to relax. She, for example, would be prescribed one, two or three times daily azelastine (or salt thereof, such as HCl) in an amount in the range of 1-8 mg and sertraline (or salt thereof, such as HCl) in an amount of 25 mg for a period of time in the range of about 1-12 weeks or longer, such as 1 mg azelastine and 25 mg sertraline for a period of about 1-4 weeks. If the patient shows improvement, she can remain on this dosing indefinitely. If any of the anxiety, trouble concentrating, trouble falling asleep, trembling, twitching, tense muscles, headaches, grouchiness, sweating, hot flashes, lightheadedness, trouble breathing, nausea, fatigue, and/or being unable to relax, or her GAD symptoms persist, the amount of the azelastine dose could be increased to 2 mg azelastine and 25 mg sertraline for a period of 1-4 weeks. If the patient shows improvement, she can remain on this dosing indefinitely. If any of the anxiety, trouble concentrating, trouble falling asleep, trembling, twitching, tense muscles, headaches, grouchiness, sweating, hot flashes, lightheadedness, trouble breathing, nausea, fatigue, and/or being unable to relax, or her GAD symptoms persist, the amount of the azelastine dose could be increased to 4 mg azelastine and 25 mg sertraline for a period of 1-4 weeks. If the patient shows improvement, she can remain on this dosing indefinitely. If any of the anxiety, trouble concentrating, trouble falling asleep, trembling, twitching, tense muscles, headaches, grouchiness, sweating, hot flashes, lightheadedness, trouble breathing, nausea, fatigue, and/or being unable to relax, or her GAD symptoms persist, the amount of the azelastine dose could be increased to 6 mg azelastine and 25 mg sertraline for a period of 1-4 weeks. If the patient shows improvement, she can remain on this dosing indefinitely. If any of the anxiety, trouble concentrating, trouble falling asleep, trembling, twitching, tense muscles, headaches, grouchiness, sweating, hot flashes, lightheadedness, trouble breathing, nausea, fatigue, and/or being unable to relax, or her GAD symptoms persist, the amount of the azelastine dose could be increased again to 8 mg azelastine and 25 mg sertraline for a period of 1-4 weeks. If the patient shows improvement, she can remain on this dosing indefinitely. Or a practitioner can start at any combination dosage of azelastine and sertraline, increase, decrease or change the dosage as needed, and when an effective dose is established, the patient can continue treatment at the effective dose.

### EXAMPLE 16

A female patient is diagnosed with premenstrual dysphoric disorder (PMDD). The patient's complaints may include one or more of: depressed mood, sadness, hopelessness, feelings of worthlessness, anxiety, tension, mood swings, irritability, anger, inability to concentrate, fatigue, changes in appetite, excessive sleeping, difficulty sleeping, breast tenderness, headaches, joint and/or muscle aches, bloating, and/or weight gain. She, for example, would be prescribed a daily amount of azelastine (or salt thereof, such as HCl) in an amount in the range of 1-6 mg and sertraline (or salt thereof, such as HCl) in an amount in the range of 25-100 mg for a period of time in the range of about 1-12 weeks or longer, such as 1 mg azelastine and 25 mg sertraline for a period of 1-4 weeks. If the patient shows improvement, she can remain on this dosing indefinitely. If any of her depressed mood, sadness, hopelessness, feelings of worthlessness, anxiety, tension, mood swings, irritability, anger, inability to concentrate, fatigue, changes in appetite, excessive sleeping, difficulty sleeping, breast tenderness, headaches, joint and/or muscle aches, bloating, and/or weight gain, or her PMDD symptoms persist, the amount of the azelastine dose could be increased to 2 mg azelastine and 25 mg sertraline for a period of 1-4 weeks. If the patient shows improvement, she can remain on this dosing indefinitely. If any of her depressed mood, sadness, hopelessness, feelings of worthlessness, anxiety, tension, mood swings, irritability, anger, inability to concentrate, fatigue, changes in appetite, excessive sleeping, difficulty sleeping, breast tenderness, headaches, joint and/or muscle aches, bloating, and/or weight gain, or her PMDD symptoms persist, the dose (azelastine and sertraline) could be increased to 4 mg azelastine and 50 mg sertraline for a period of 1-4 weeks. If the patient shows improvement, she can remain on this dosing indefinitely. If any of her depressed mood, sadness, hopelessness, feelings of worthlessness, anxiety, tension, mood swings, irritability, anger, inability to concentrate, fatigue, changes in appetite, excessive sleeping, difficulty sleeping, breast tenderness, headaches, joint and/or muscle aches, bloating, and/or weight gain, or her PMDD symptoms persist, the dose (azelastine and sertraline) could be increased to 6 mg azelastine and 100 mg sertraline for a period of 1-4 weeks. If the patient shows improvement, she can remain on this dosing indefinitely. If any of her depressed mood, sadness, hopelessness, feelings of worthlessness, anxiety, tension, mood swings, irritability, anger, inability to concentrate, fatigue, changes in appetite, excessive sleeping, difficulty sleeping, breast tenderness, headaches, joint and/or muscle aches, bloating, and/or weight gain, or her PMDD symptoms persist, the amount of the azelastine dose could be increased to 8 mg azelastine and 100 mg sertraline for a period of 1-4 weeks. If the patient shows improvement, she can remain on this dosing indefinitely. Or a practitioner can start at any combination dosage of azelastine and sertraline, increase, decrease or change the dosage as needed, and when an effective dose is established, the patient can continue treatment at the effective dose.

### EXAMPLE 17

A female patient is diagnosed with premenstrual dysphoric disorder (PMDD). The patient's complaints may include one or more of: depressed mood, sadness, hopelessness, feelings of worthlessness, anxiety, tension, mood swings, irritability, anger, inability to concentrate, fatigue, changes in appetite, excessive sleeping, difficulty sleeping, breast tenderness, headaches, joint and/or muscle aches, bloating, and/or weight gain. She, for example, would be prescribed a daily amount of azelastine (or salt thereof, such as HCl) in an amount in the range of 1-8 mg and sertraline (or salt thereof, such as HCl) in an amount in the range of 25-200 mg for a period of time in the range of about 1-12 weeks or longer, such as 1 mg azelastine and 25 mg sertraline for a period of 1-4 weeks. If the patient shows improvement, she can remain on this dosing indefinitely. If any of the depressed mood, sadness, hopelessness, feelings of worthlessness, anxiety, tension, mood swings, irritability, anger, inability to concentrate, fatigue, changes in appetite, excessive sleeping, difficulty sleeping, breast tenderness, headaches, joint and/or muscle aches, bloating, and/or weight gain, or her PMDD symptoms persist, the dose (azelastine and sertraline) could be increased to 2 mg azelastine and 50 mg sertraline for a period of 1-4 weeks. If the patient shows improvement, she can remain on this dosing indefinitely. If any of the depressed mood, sadness, hopelessness, feelings of worthlessness, anxiety, tension, mood swings, irritability, anger, inability to concentrate, fatigue, changes in appetite, excessive sleeping, difficulty sleeping, breast tenderness, headaches, joint and/or muscle aches, bloating, and/or weight gain, or her PMDD symptoms persist, the dose (azelastine and sertraline) could be increased to 4 mg azelastine and 100 mg sertraline for a period of 1-4 weeks. If the patient shows improvement, she can remain on this dosing indefinitely. If any of the depressed mood, sadness, hopelessness, feelings of worthlessness, anxiety, tension, mood swings, irritability, anger, inability to concentrate, fatigue, changes in appetite, excessive sleeping, difficulty sleeping, breast tenderness, headaches, joint and/or muscle aches, bloating, and/or weight gain, or her PMDD symptoms persist, the dose (azelastine and sertraline) could be increased to 6 mg azelastine and 150 mg sertraline for a period of 1-4 weeks. If the patient shows improvement, she can remain on this dosing indefinitely. If any of the depressed mood, sadness, hopelessness, feelings of worthlessness, anxiety, tension, mood swings, irritability, anger, inability to concentrate, fatigue, changes in appetite, excessive sleeping, difficulty sleeping, breast tenderness, headaches, joint and/or muscle aches, bloating, and/or weight gain, or her PMDD symptoms persist, the dose (azelastine and sertraline) could be increased to 8 mg azelastine and 200 mg sertraline for a period of 1-4 weeks. If the patient shows improvement, she can remain on this dosing indefinitely. Or a practitioner can start at any combination dosage of azelastine and sertraline, increase, decrease or change the dosage as needed, and when an effective dose is established, the patient can continue treatment at the effective dose.

### EXAMPLE 18

A patient diagnosed with mild chronic depression (MCD) with constant sadness and lack of interest and feeling lonely and helpless, for example, would be prescribed a daily amount of azelastine (or salt thereof, such as HCl) in an amount in the range of 1-4 mg and sertraline (or salt thereof, such as HCl) in an amount in the range of 25-50 mg for about 1-12 weeks or longer, such as 1 mg azelastine and 50 mg sertraline for 4 weeks (could be administered up to 3×/day). If the patient shows improvement, they can remain on this dosing indefinitely. If any of the sadness and lack of interest and feeling lonely and helpless, or MCD symptoms persist, the dose could be changed (e.g., increase the amount of the azelastine dose and decrease the amount of the sertraline dose) to 2 mg azelastine and 25 mg sertraline for a period of 2-4 weeks. If the patient shows improvement, they can remain on this dosing indefinitely. If any of the sadness and lack of interest and feeling lonely and helpless, or the MCD symptoms persist, the dose could be changed (by increasing the azelastine dose) to 4 mg azelastine and 25 mg sertraline for a period of 2-4 weeks. If the patient shows improvement, they can remain on this dosing indefinitely. If any of the sadness and lack of interest and feeling lonely and helpless, or the MCD symptoms persist, the dose could be changed (by increasing the amount of both the azelastine dose and the sertraline dose) to 6 mg azelastine and 50 mg sertraline for 2-4 weeks. If the patient shows improvement, they can remain on this dosing indefinitely. Or a practitioner can start at any combination dosage of azelastine and sertraline, increase, decrease or change the dosage as needed, and when an effective dose is established, the patient can continue treatment at the effective dose.

### EXAMPLE 19

A patient diagnosed with mild chronic depression (MCD) with constant sadness and lack of interest and feeling lonely and helpless, for example, would be prescribed a daily amount of azelastine (or salt thereof, such as HCl) in an amount in the range of 1-2 mg and sertraline (or salt thereof, such as HCl) in an amount in the range of 150-200 mg for a period of time in the range of about 1-12 weeks or longer, such as 1 mg azelastine and 150 mg sertraline for 4 weeks. If the patient shows improvement, they can remain on this dosing indefinitely. If any of the sadness and lack of interest and feeling lonely and helpless, or MCD symptoms persist, the dose could be changed (by increasing the amount of the sertraline dose) to 1 mg azelastine and 200 mg sertraline for a period of 2-4 weeks. If the patient shows improvement, they can remain on this dosing indefinitely. If any of the sadness and lack of interest and feeling lonely and helpless, or MCD symptoms persist, the dose could be changed (by increasing the amount of azelastine) to 2 mg azelastine and 200 mg sertraline for a period of 2-4 weeks. If the patient shows improvement, they can remain on this dosing indefinitely. Or a practitioner can start at any combination dosage of azelastine and sertraline, increase, decrease or change the dosage as needed, and when an effective dose is established, the patient can continue treatment at the effective dose.

### EXAMPLE 20

A patient diagnosed with mild chronic depression (MCD) with constant sadness and lack of interest and feeling lonely and helpless, for example, would be prescribed a daily amount of azelastine (or salt thereof, such as HCl) in an amount in the range of 6-8 mg and sertraline (or salt thereof, such as HCl) in an amount in the range of 25-150 mg for a period of time in the range of about 1-12 weeks or longer, such as 6 mg azelastine and 25 mg sertraline for 4 weeks. If the patient shows improvement, they can remain on this dosing indefinitely. If any of the sadness and lack of interest and feeling lonely and helpless, or MCD symptoms persist, the dose could be changed (by increasing the sertraline dose) to 6 mg azelastine and 100 mg sertraline for a period of 2-4 weeks. If the patient shows improvement, they can remain on this dosing indefinitely. If any of the sadness and lack of interest and feeling lonely and helpless, or MCD symptoms persist, the dose could be changed (by increasing the amount of the azelastine dose and decreasing the amount of the sertraline dose) to 8 mg azelastine and 25 mg sertraline for a period of 2-4 weeks. If the patient shows improvement, they can remain on this dosing indefinitely. If any of the sadness and lack of interest and feeling lonely and helpless, or MCD symptoms persist, the dose could be changed (by increasing the amount of the sertraline dose) to 8 mg azelastine and 50 mg sertraline for 2-4 weeks. If the patient shows improvement, they can remain on this dosing indefinitely. If any of the sadness and lack of interest and feeling lonely and helpless, or MCD symptoms persist, the dose could be changed (by further increasing the amount of the sertraline dose) to 8 mg azelastine and 150 mg sertraline for a period of about 2-4 weeks. If the patient shows improvement, they can remain on this dosing indefinitely. Or a practitioner can start at any combination dosage of azelastine and sertraline, increase, decrease or change the dosage as needed, and when an effective dose is established, the patient can continue treatment at the effective dose.

### EXAMPLE 21

A patient diagnosed with depression and having excessive worries, persistent sadness and anxiety, feeling exhausted and having no sex drive, for example, would be prescribed a daily amount of azelastine (or salt thereof, such as HCl) in an amount in the range of 1-6 mg and sertraline (or salt thereof, such as HCl) in an amount in the range of 25-200 mg for a period of time in the range of about 1-12 weeks or longer, such as 1 mg azelastine and 25 mg sertraline (could be administered one, two or three times daily) for up to a period of about 2-4 weeks. If the patient shows improvement, they can remain on this dosing indefinitely. If any of the excessive worries, persistent sadness and anxiety, feeling exhausted and having no sex drive, or depression symptoms persist, the dose could be changed (by increasing the amount of both) to 4 mg azelastine and 50 mg sertraline for 2-4 weeks. If the patient shows improvement, they can remain on this dosing indefinitely. If any of the excessive worries, persistent sadness and anxiety, feeling exhausted and having no sex drive, or depression symptoms persist, the dose could be changed (by increasing azelastine and decreasing sertraline) to 6 mg azelastine and 25 mg sertraline for 2-4 weeks. If the patient shows improvement, they can remain on this dosing indefinitely. If any of the excessive worries, persistent sadness and anxiety, feeling exhausted and having no sex drive, or depression symptoms persist, the dose could be changed (by increasing sertraline) to 6 mg azelastine and 150 mg sertraline for 2-4 weeks. If the patient shows improvement, they can remain on this dosing indefinitely. If any of the excessive worries, persistent sadness and anxiety, feeling exhausted and having no sex drive, or depression symptoms persist, the dose could be changed (by decreasing azelastine and increasing sertraline) to 1 mg azelastine and 200 mg sertraline for 2-4 weeks. If the patient shows improvement, they can remain on this dosing indefinitely. Or a practitioner can start at any combination dosage of azelastine and sertraline, increase, decrease or change the dosage as needed, and when an effective dose is established, the patient can continue treatment at the effective dose.

### EXAMPLE 22

A patient diagnosed with depression and having excessive worries, persistent sadness and anxiety, feeling exhausted and having no sex drive, for example, would be prescribed a daily amount of azelastine (or salt thereof, such as HCl) in an amount in the range of 4-8 mg and sertraline (or salt thereof, such as HCl) in an amount in the range of 25-200 mg for a period of time in the range of about 1-12 weeks or longer, such as 4 mg azelastine and 25 mg sertraline (could be administered 2×/day) for 2-4 weeks. If the patient shows improvement, they can remain on this dosing indefinitely. If any of the excessive worries, persistent sadness and anxiety, feeling exhausted and having no sex drive, or depression symptoms persist, the dose could be changed (by increasing azelastine) to 8 mg azelastine and 25 mg sertraline for 2-4 weeks. If the patient shows improvement, they can remain on this dosing indefinitely. If any of the excessive worries, persistent sadness and anxiety, feeling exhausted and having no sex drive, or depression symptoms persist, the dose could be changed (by decreasing azelastine and increasing sertraline) to 6 mg azelastine and 50 mg sertraline for 2-4 weeks. If the patient shows improvement, they can remain on this dosing indefinitely. If any of the excessive worries, persistent sadness and anxiety, feeling exhausted and having no sex drive, or depression symptoms persist, the dose could be changed (by increasing sertraline) to 6 mg azelastine and 100 mg sertraline for 2-4 weeks. If the patient shows improvement, they can remain on this dosing indefinitely. If any of the excessive worries, persistent sadness and anxiety, feeling exhausted and having no sex drive, or depression symptoms persist, the dose could be changed (by increasing both) to 8 mg azelastine and 200 mg sertraline for 2-4 weeks. If the patient shows improvement, they can remain on this dosing indefinitely. Or a practitioner can start at any combination dosage of azelastine and sertraline, increase, decrease or change the dosage as needed, and when an effective dose is established, the patient can continue treatment at the effective dose.

### EXAMPLE 23

A patient diagnosed with anxiety and having uncontrollable and persistent worry, fatigue, headaches, irritability, and nervousness, feelings of worry, nausea, headaches, sweating, muscle aches, and difficulty of falling asleep, for example, would be prescribed a daily amount of azelastine (or salt thereof, such as HCl) in an amount in the range of 1-8 mg and sertraline (or salt thereof, such as HCl) in an amount in the range of 25-200 mg for a period of time in the range of about 1-12 weeks or longer, such as 1 mg azelastine and 25 mg sertraline for 2-4 weeks (could be administered up to 4×/day). If the patient shows improvement, they can remain on this dosing indefinitely. If any of the uncontrollable and persistent worry, fatigue, headaches, irritability, and nervousness, feelings of worry, nausea, headaches, sweating, muscle aches, and difficulty of falling asleep, or anxiety persist, the dose could be changed (by increasing both) to 2 mg azelastine and 100 mg sertraline for 2-4 weeks. If the patient shows improvement, they can remain on this dosing indefinitely. If any of the uncontrollable and persistent worry, fatigue, headaches, irritability, and nervousness, feelings of worry, nausea, headaches, sweating, muscle aches, and difficulty of falling asleep, or anxiety persist, the dose could be changed (by decreasing azelastine and increasing sertraline) to 1 mg azelastine and 150 mg sertraline for 2-4 weeks. If the patient shows improvement, they can remain on this dosing indefinitely. If any of the uncontrollable and persistent worry, fatigue, headaches, irritability, and nervousness, feelings of worry, nausea, headaches, sweating, muscle aches, and difficulty of falling asleep, or anxiety persist, the dose could be changed (by increasing azelastine and decreasing sertraline) to 4 mg azelastine and 50 mg sertraline for 2-4 weeks. If the patient shows improvement, they can remain on this dosing indefinitely. If any of the uncontrollable and persistent worry, fatigue, headaches, irritability, and nervousness, feelings of worry, nausea, headaches, sweating, muscle aches, and difficulty of falling asleep, or anxiety persist, the dose could be changed (by increasing both) to 6 mg azelastine and 100 mg sertraline for 2-4 weeks. If the patient shows improvement, they can remain on this dosing indefinitely. If any of the uncontrollable and persistent worry, fatigue, headaches, irritability, and nervousness, feelings of worry, nausea, headaches, sweating, muscle aches, and difficulty of falling asleep, or anxiety persist, the dose could be changed (by increasing azelastine and decreasing sertraline) to 8 mg azelastine and 25 mg sertraline for 2-4 weeks. If the patient shows improvement, they can remain on this dosing indefinitely. If any of the uncontrollable and persistent worry, fatigue, headaches, irritability, and nervousness, feelings of worry, nausea, headaches, sweating, muscle aches, and difficulty of falling asleep, or anxiety persist, the dose could be changed (by increasing sertraline) to 8 mg azelastine and 200 mg sertraline for 2-4 weeks. If the patient shows improvement, they can remain on this dosing indefinitely. Or a practitioner can start at any combination dosage of azelastine and sertraline, increase, decrease or change the dosage as needed, and when an effective dose is established, the patient can continue treatment at the effective dose.

### REFERENCES

US Patent documents: U.S. Pat. No. 8,865,733, Oct. 21, 2014, Felder; U.S. Pat. No. 9,919,050, Mar. 20, 2018, Dang, et al; U.S. Pat. No. 8,758,816, Jun. 24, 2014, Fuge, et al; U.S. Pat. No. 8,168,620, May 1, 2012, Lulla, et al; U.S. Pat. No. 7,022,687, Apr. 4, 2006, Szelenyl, et al; U.S. Pat. No. 5,086,050, Feb. 4, 1992, Hettche, et al; U.S. Pat. No. 5,068,233, Nov. 26, 1991, Achterrath-Tuckerman, et al.

Rosenblat J D, Kakar R, McIntyre R S, 2015. Int J Neuropsychopharmacol, The Cognitive Effects of Antidepressants in Major Depressive Disorder: A Systematic Review and Meta-Analysis of Randomized Clinical Trials. July 25; 19(2).

Ewgeni Jakubovski, Anjali L Varigonda, Nicholas Freemantle, Matthew J Taylor, 2016. Systematic Review and Meta-Analysis: Dose-Response Relationship of Selective Serotonin Reuptake Inhibitors in Major Depressive Disorder. Am J Psychiatry, 173(2): 174-83.

Raza Sagarwala. 2019. Changes in Inflammatory Biomarkers Before and after SSRI Therapy in PTSD: A Review. Ann Clin Psychiatry, 31(4): 292-297.

J A Fawcett, H M Kravitz, 1982. Sertraline: Pharmacokinetics, Clinical Efficacy, and Mechanism of Action. Pharmacotherapy, 2(5 ): 243-54.

Ruihua Hou, 2017, Peripheral inflammatory cytokines and immune balance in Generalized Anxiety Disorder: case-controlled study. Brain Behav Immun. 2017 May; 62: 212-218.

Michelle Guignet, 2020, Persistent behavior deficits, neuroinflammation, and oxidative stress in a rat model of acute organophosphate intoxication, Vol 133, January 2020, 104431.

Chun-Hong Liu, 2019, Role of inflammation in Depression Relapse, Journal of Neuroinflammation (2019) 16: 90.

Jovana Vojvodic, et al, 2019, The Impact of Immunological Factors on Depression Treatment-Relation Between Antidepressants and Immunomodulation Agents, Macedonian Journal of Medical Sciences. 2019 September 30; 7(18): 3064-3069.

F. C. Bennett and A. V. Molofsky, 2019, The Immune System and Psychiatric Disease: A Basic Science Perspective, Clinical and Experimental Immunology, 197: 294-307.

Ruihua Hou and David S. Baldwin, 2012, A Neuroimmunological Perspective on Anxiety Disorders, Human Psychopharmacol Clin Exp. Vol 27: 6-14.

N Kappelmann, G Lewis, R Dantzer, PB Jones and GM Khandaker, 2018, Antidepressant Activity of Anti-cytokine Treatment: a Systematic Review and Meta-analysis of Clinical Trials of Chronic Inflammatory Conditions, Molecular Psychiatry. Vol. 23, 335-343.

A. Atri, S. D. Rountree, O. L. Lopez, and R. S. Doody, 2018, Impact of Mast Cells in Depression Disorder: Inhibitory Effect of IL-37 (New Frontiers). Immunol Res, vol. 66(3 ), 323-331 Jun 2018.

Sung Ho Maeng and Heeok Hong, 2019, Inflammation as the Potential Basis in Depression. Int Neurourol J 2019; Vol 23(Suppl 2): S 63-71.

Ole Köhler, et al. van Hoven PT, Kaufman A, Carr WW. Inflammation in Depression and the Potential for Anti-Inflammatory Treatment. Current Neuropharmacology, 2016, 14, 732-742.

Anzela Niraula et al. 2019, Interleukin- 6 Induced by Social Stress Promotes a Unique Transcriptional Signature in the Monocytes That Facilitate Anxiety. Biol Psychiatry 85(8 ), 679-689 2019 April 15.

M Catena-Dell'Osso, et al, 2011, Inflammatory and Neurodegenerative Pathways in Depression: A New Avenue for Antidepressant Development? Curr Med Chem. 18(2 ), 245-55.

S Georgin-Lavialle et al. 2016, Mast Cells' Involvement in Inflammation Pathways Linked to Depression: Evidence in Mastocytosis. Mol Psychiatry. 21(11 ), 1511-1516 Nov 2016.

Romain Troubat et al, 2020, Neuroinflammation and Depression: A Review. Eur J Neurosci. 2020 Mar. 9 DOI: 10.1111/ejn.14720.

Ja Wook Kooa, et al, 2010, Nuclear factor-KB is a critical mediator of Stress Impaired Neurogenesis and Depressive Behavior. PNAS, Feb. 9, 2010, Vol. 107(6 ) 2669-2674.

Patricia B Williams, Elizabeth Crandall and John D Sheppard, 2010, Azelastine Hydrochloride, a Dual-acting Anti-inflammatory Ophthalmic Solution For Treatment of Allergic Conjunctivitis. Clinical Ophthalmology 2010: 4 993-1001.

Hazama H, Nakajima T, Hisada T, Hamada E, Omata M, Kurachi Y. Effects of azelastine on membrane currents in tracheal smooth muscle cells isolated from the guinea-pig. Eur J Pharmacol. 1994; 259: 143-150.

Szelenyi I, Achterrath-Tuckermann U, Schmidt J, Minker E, Paegelow I, Werner H. Azelastine: A multifaceted drug for asthma therapy. Agents Actions Suppl. 1991; 34: 295-311.

Galatowicz G, Ajayi Y, Stern M E, Calder V L. Ocular antiallergic compounds selectively inhibit human mast cell cytokines in vitro and conjunctival cell infiltration in vivo. Clin Exp Allergy. 2007; 37: 1648-1656.

Ciprandi G, Pronzato C, Passalacqua G, et al. Topical azelastine reduces eosinophil activation and intercellular adhesion molecule-1 expression on nasal epithelial cells: An antiallergic activity. J Allergy Clin Immunol. 1996; 98(6 Pt 1): 1088-1096.

Simons F E, Simons K J. Clinical pharmacology of new histamine H1 receptor antagonist. Clin Pharmacokinet. 1999; 36: 329-352.

Aiko Hatakeyama, 2008, Azelastine hydrochloride on behavioral and psychological symptoms and activities of daily living in dementia patients. Geriatr Gerontol Int 2008; 8: 59-61.

Duraisamy Kempuraj, et al. 2003, Azelastine Inhibits Secretion of IL-6, TNF-alpha and IL-8 as Well as NF-kappaB Activation and Intracellular Calcium Ion Levels in Normal Human Mast Cells. Int Arch Allergy Immunol. 132(3 ), 231-9 November 2003.

Kazunori Yoneda, et al. 1997, Suppression by Azelastine Hydrochloride of NF-KB Activation Involved in Generation of Cytokines and Nitric Oxide. Japanese Journal of Pharmacology, 73: 145-53.

Loyd Allen, Ansel's Pharmaceutical Dosage Forms and Drug Delivery Systems, Tenth (2013).

Sarfaraz K. Niazi, Handbook of Pharmaceutical Manufacturing Formulations Volumes 1-6.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Any of the methods disclosed herein can be used with any of the compositions disclosed herein or with any other compositions. Likewise, any of the disclosed compositions can be used with any of the methods disclosed herein or with any other methods. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A pharmaceutical composition, comprising:
azelastine; and
sertraline;
and one or more pharmaceutically acceptable excipients, wherein the pharmaceutical composition comprises effective amounts of the azelastine and the sertraline which together treat anxiety, depression, major depressive disorder, generalized anxiety disorder, mild chronic depression, premenstrual dysphoric disorder or anxiety-related sleep disorder of a patient.

2. The pharmaceutical composition of claim 1, wherein the azelastine is present in the pharmaceutical composition in an amount in a range of about 1 mg to about 8 mg.

3. The pharmaceutical composition of claim 1, wherein the sertraline is present in the pharmaceutical composition in an amount in a range of about 25 mg to about 200 mg.

4. The pharmaceutical composition of claim 2, wherein the sertraline is present in the pharmaceutical composition in an amount in a range of about 25 mg to about 200 mg.

5. The pharmaceutical composition of claim 1, wherein:
the azelastine is present in the pharmaceutical composition in an amount in the range of about 1 mg to about 6 mg; and
the sertraline is present in the pharmaceutical composition in an amount in the range of about 25 mg to about 200 mg.

6. The pharmaceutical composition of claim 1, wherein the pharmaceutical composition is formulated as an oral pharmaceutical dosage form.

7. The pharmaceutical composition of claim 6, wherein the oral pharmaceutical dosage form is a solid form or a liquid form.

8. A method comprising:
administering the pharmaceutical composition of claim 1 to the patient to treat anxiety, depression, major depressive disorder, generalized anxiety disorder, mild chronic depression, premenstrual dysphoric disorder or anxiety-related sleep disorder of the patient.

9. The method of claim 8, wherein the azelastine and the sertraline are present in the pharmaceutical composition in synergistic amounts effective for treating major depressive disorder.

10. The method of claim 8, wherein the pharmaceutical composition is administered once or twice a day, or once every 2 or 3 or 4 days to the patient in an oral solid or liquid form.

11. The method of claim 10, wherein the pharmaceutical composition is administered for a period of at least 4 weeks.

12. The method of claim 8, wherein the azelastine is present in the pharmaceutical composition in an amount in a range of about 1 mg to about 8 mg.

13. The method of claim 8, wherein the sertraline is present in the pharmaceutical composition in an amount in a range of about 25 mg to about 200 mg.

14. The method of claim 8, wherein the sertraline is present in the pharmaceutical composition in an amount in a range of about 50 mg to about 150 mg.

15. The method of claim 8, wherein the azelastine is present in the pharmaceutical composition in an amount in a range of about 2 mg to about 6 mg.

16. The method of claim 8, wherein:
the azelastine is present in the pharmaceutical composition in an amount in a range of about 2 mg to about 6 mg; and
the sertraline is present in the pharmaceutical composition in an amount in range of about 50 mg to about 150 mg.
